(19) United States
(12) Patent Application Publication
(10) Pub. No.: US 2006/0167537 A1
(43) Pub. Date: Jul. 27, 2006

(54) Stent assembly and device for application thereof

(76) Inventors: Rolf Larsson, Blomstervagen (SE); Carl-Gustaf Groth, Skoldvagen (SE)

Correspondence Address: YOUNG & THOMPSON, 745 SOUTH 23RD STREET, 2ND FLOOR, ARLINGTON, VA, 22202, US

(21) Appl. No.: 10/510,285

(22) PCT Filed: Apr. 4, 2003
(86) PCT No.: PCT/SE03/00545

§ 371 Date: Jul. 19, 2005

(30) Foreign Application Priority Data
Apr. 8, 2002 (SE) 0201053-6

(51) Int. Cl. A61F 2/84; A61F 2/92

(52) U.S. Cl. 623/1.13; 623/1.44; 623/1.11

(57) ABSTRACT

A stent assembly (2) includes a stent capable of assuming a contracted and an expanded configuration. A membrane of a biocompatible material impermeable to molecular transport across the membrane is wrapped around the stent more than one full turn. There is also disclosed a device for performing anastomosis, including a stent assembly (2) in a contracted state and a holding and gripping device (12). The handle has a handle portion (14) and a support structure (16, 18) for accommodating the stent assembly. There is a locking loop (20) that can be tightened around the stent assembly when it is positioned on the support structure, and released to allow the stent assembly to expand to a nominal size.

The present invention relates generally to devices and methods for connecting severed vessels, e.g. arteries and veins, and in particular to a covered stent and a method and system for applying the covered stent.

### BACKGROUND OF THE INVENTION

Currently employed techniques for connecting blood vessels in the context of transplantation surgery and other similar kinds of surgical procedures are based on suturing the ends of the vessel with a continuous suture. This is a time consuming procedure that often may require using a microscope and is rendered complicated by limited accessibility to the site of surgery in the body. There are suggestions in the patent literature for facilitating and simplifying this procedure, but they nevertheless suffer from lack of ability to meet some essential requirements. Namely, the method should

be easy to perform in a short time

provide immediate and efficient sealing under physiological pressure and temperature conditions

maintain patency (i.e. should not induce trombosis or inflammation)

be user friendly

provide devices suitable for industrial production

The patent literature comprises a vast number of patents relating to stents and devices for manipulating stents.

One example of a prior art stent comprising shape memory is disclosed i.a. in U.S. Pat. No. 5,354,308 (Simon et al). It relates to a stent comprising a wire skeletal frame, said frame being adapted to assume a first condition in which said frame is expanded, and a second contracted condition. The wire frame comprises a metallic compound of nickel and titanium, said compound in said second condition retaining memory of said first condition. When heated to a selected body temperature, it assumes said first condition in which said frame is greatly expanded relative to said second condition.

### SUMMARY OF THE INVENTION

The object of the present invention is to provide an improved device that meets the desired requirements outlined above, in particular to provide immediate and efficient sealing under physiological pressure and temperature conditions in combination with providing long lasting patency. This object is achieved in a first aspect with a device as defined in claim 1.

Thereby a stent, capable of assuming a contracted and an expanded configuration, and preferably made of a shape memory material, is provided in a reduced size and a piece or membrane of foil material, e.g. polymer, is tightly wrapped around the stent as a separate entity. Thus, the ends of said membrane may either be loose, i.e. they are not attached or connected to the stent in any way, or the inner end part of the membrane may be fixed to the stent at a small fraction of the circumference of the stent. This means that if the stent is allowed to expand to its nominal size, the membrane will follow in this expansion, and accommodate to the varying diameter. Preferably, the length of the membrane is selected such that in a fully expanded state of the stent, the ends of the polymer membrane or foil will overlap only by a fraction of the circumference, i.e. the membrane is wrapped around the stent at least slightly more than one full turn.

In order to provide and ascertain complete blood compatibility, the surfaces of those parts that come into contact with body fluids inside the vessel in which they are located, will preferably be treated chemically or biologically. The preferred treatment is to coat the surfaces with heparin, although other surface treatments are also possible and within the inventive concept.

Also, it should allow a stent to be applied or introduced by a simple and quick method. Thus, in a further aspect there is-provided a device for enabling easy application of the stent and for connecting blood vessels. This device is defined in claim 12.

### BRIEF DESCRIPTION OF THE DRAWINGS

The present invention will become more fully understood from the detailed description given hereinbelow and the accompanying drawings which are given by way of illustration only and thus not limitative of the present invention, and wherein

FIG. 1 is an illustration of a stent in general (not according to the invention);

FIG. 2a illustrates an embodiment of the device according to the invention wherein a stent is provided in a contracted state;

FIG. 2b illustrates the same device as in FIG. 2a but wherein the stent has been released and expanded to a nominal size;

FIG. 2b schematically illustrates a variation of the stent and membrane assembly according to the invention wherein the membrane is attached to the stent;

FIG. 3 is a perspective view of a device for anastomosis comprising a stent/membrane assembly as shown in FIG. 2;

FIG. 4 illustrates an embodiment of the membrane of the assembly;

FIG. 5a shows an embodiment of end stops according to the invention;

FIG. 5b shows an alternative embodiment of end stops;

FIG. 5c shows a cross-section through a blood vessel with a stent as in FIG. 5a;

FIG. 6 shows an embodiment where the membrane is secured with a glue dot; and

FIG. 7a-b are cross-sections through a stent and balloon catheter assembly in non-inflated and inflated conditions, respectively.

### DETAILED DESCRIPTION OF PREFERRED EMBODIMENTS

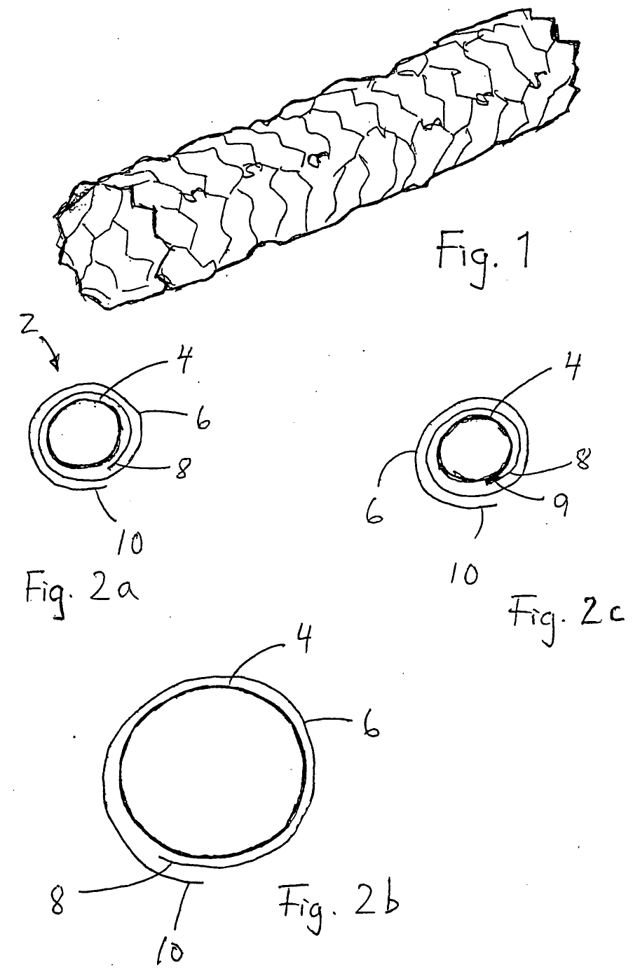

Stents for anastomosis are supplied in numerous designs, and FIG. 1 illustrates one type of stent suitable for use in the present invention. It comprises a tubular member made from expandable shape memory metal. An example of a suitable material is NiTinol™, which has the capability to regain a given shape when exposed to a given temperature. For the purposes of the present invention, it should be capable of assuming a desired shape when exposed to body temperature, e.g. about 37° C.

Stents of this type are commercially available, e.g. under the trade name SelfX from Jomed A B, Helsingborg, Sweden. Thus, the actual specific design of the stent member as such does not form part of the present invention.

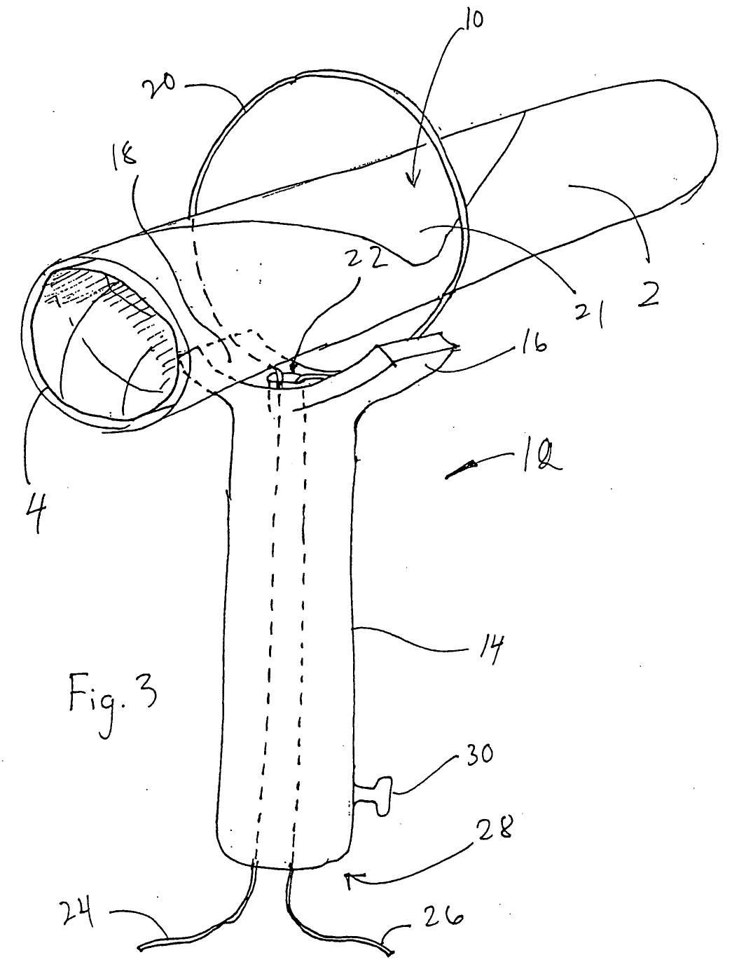

FIG. 2a-c illustrate embodiments of the basic idea behind the invention.

A problem often encountered in anastomosis is that the finished joint is very difficult to make absolutely leak-proof under the physiological conditions of pressure prevailing in blood vessels. Therefore, according to the invention a stent has been provided with means for completely sealing the joint or seam between the two ends of the blood vessels on which the anastomosis has been carried out. Thus, a stent assembly 2 according to the present invention comprises a stent member 4, as described above and capable of assuming a contracted and an expanded configuration, and a piece or sheet/membrane 6 made of a material that is biocompatible and impermeable to liquids and molecular transport, tightly wrapped around the stent member when the stent is in a contracted state. Thus, at least one end 10 of said membrane in this embodiment is loose, i.e. it is not attached or connected to the stent in any way. This means that if the stent is allowed to expand to its nominal size (see FIG. 2b), the membrane will follow in this expansion. Preferably, the length of the membrane is selected such that in a fully expanded state of the stent, the ends of the polymer membrane or foil will overlap only by a fraction of the circumference. This implies that the membrane will be wrapped a few turns around the stent in its contracted state. Thereby the membrane will exert two functions: i) by virtue of the total thickness of the coiled membrane, it will offer enough resistance to prevent premature expansion of the stent: and ii) after expansion of the stent inside a blood vessel, the membrane will be provided essentially as a mono-layer, ends overlapping by only a few mm, in a standard size of the stent by say 5-10 mm. The degree of overlapping is not critical, and will of course also depend on the size of the stent.

An important advantage connected with using a non-porous membrane is that it efficiently separates the fresh anastomosis joint from the circulating blood. This joint would otherwise inevitably lead to activation which can trigger a number of processes causing problems, such as thrombosis, cell proliferation or hyperplacia.

In another embodiment the inner end 8 of the membrane contacting the surface of the stent can be attached 9 to said surface, see FIG. 2c, by gluing, welding or other suitable method.

The material for the membrane is suitably a polymer material, but other materials are possible as long as they are biocompatible and meet the requirement of impermeability. Preferably the material can be a laminated material wherein the two layers-have slightly different properties in terms of thermal expansion coefficients. This means that when subjecting the membrane to a temperature change, the upper and lower layers will expand or contract at a slightly different rate. This will cause the membrane to become slightly curved. In such a condition it is easier to wrap the membrane around a stent. Furthermore, if the curvature of the membrane can be made to be smaller than the curvature of the stent, at least in the expanded (nominal) condition of the stent, the membrane will accommodate much better to the circumference of the stent, and there will be no tendency for the membrane to spring off or away from the surface of the stent. It can be advantageous to apply a cold spray to the membrane when it has been mounted on the stent, since the different thermal coefficients of the layers will cause the membrane to tighten further around the stent.

A suitable laminate film meeting the above requirements is a laminate of polyethylene/polyester (e.g. Steriking ESE 1240 from Wipak, Nastola, Finland). Biodegradable materials are also possible and could be of benefit in certain circumstances.

The basic idea is that a contracted stent having an impermeable sealing membrane wrapped around it is provided. The contracted state of the stent is achieved by simply cooling it, e.g. by a cold spray. The contracted state is preserved by providing some clamping means around the assembly of stent/membrane. A presently preferred embodiment of a device for anastomosis 12 according to the invention is shown in FIG. 3.

Thus, the device 2 according to the invention is provided with a handle 14 (or manipulator piece), having a support structure 16 for the stent/membrane assembly 2 to rest in. This can be in the form of a semi-circular surface 18 having a radius of curvature corresponding to the diameter of the assembly 2. The surface 18 of the support structure preferably has a relatively limited width, i.e. it may resemble a fork, in order not to occupy too much space at the site of the blood vessel anastomosis during application of the stent. The handle 14 is preferably tube shaped or at least it has a central channel for accommodating a thread 20 (described further below), and opening in an aperture 22 in the surface 18 of the support structure 16.

The thread 20 is provided around the stent/membrane assembly 2 one single turn, to tighten the assembly and thus preventing it from unwinding. In order to further reduce the tendency of the end of the membrane to deflect from the circumference of the assembly, thereby causing potential problems during application or insertion of the assembly into a blood vessel, the outer (free) end of the membrane can be made to exhibit a triangular shape (indicated in FIG. 3), such that the tightening thread runs across the apex 21 of said triangle.

The loose ends 24, 26 of the thread 20 are passed through the above mentioned aperture 22 in the support structure 16. The thread ends 24, 26 preferably exit at the opposite end 28 of the handle 14, where they are secured by a suitable clamping means so that it is not possible for the stent assembly to inadvertently expand before it has been successfully positioned in the site where the anastomsis is to be performed. The ends can simply be tied to an anchoring element 30 by tying a knot, or alternatively there can be provided a friction engagement in the form of a slit (not shown), in which the thread ends can be clamped. Of course it is equally possible to let the thread exit anywhere else on the handle, but at present it appears most convenient to simply let the tread 20 pass through the inner lumen of the handle to its rear end 28.

The details of the locking or clamping means do not form part of the invention per se, and many other options are available, as realized by the skilled man who can easily find alternatives without inventive work.

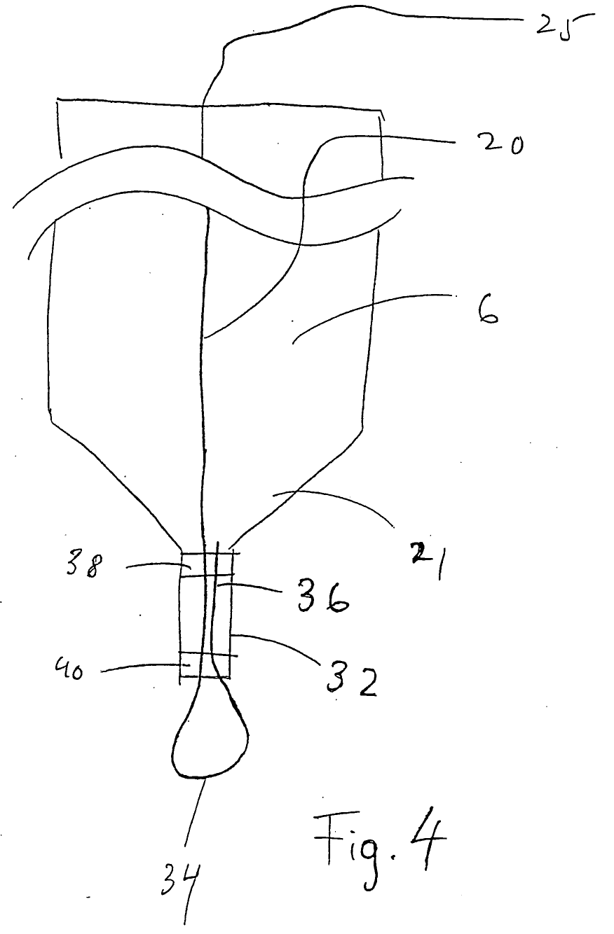

In a further embodiment of the invention the thread 20 is attached to the membrane 6 at its outer end 10, see FIG. 4. The means of attaching can be of different kinds, but a method which at present is preferred is to design the membrane so as to exhibit a tab 32 located at the apex of the triangularly shaped end portion 21. The end of the thread 20 is formed to a loop 34, and then the thread 20 is positioned on said tab 32 such that the loop 34 extends out from the tab, as shown in FIG. 4 and such that the end 36 of the thread 20 runs parallel with the thread. Then a small piece 38 of membrane material is positioned on top of the tab 32 at the inner end thereof, and another small piece 40 of membrane material is positioned on top of the tab 32 at the outer end thereof, such that the thread is sandwiched between the tab and the pieces of membrane material. Alternatively one single piece of membrane material having about the same size as the tab 32 is placed on the threads to form a sandwich assembly. Heat is then applied by pressing the sandwich structure between two heated members (e.g. two heated platens, heated pliers or the like) so as to weld the material 38, 40 pieces onto the tab 32 thereby fixating the thread on the tab. In this embodiment there will only be one thread end 25. Thus, when the membrane is wrapped around the stent in a crimped or contracted state, there will be only one single strand of thread extending through the handle 14 of the manipulator 12.

Because of the double layer of membrane material on the tab, and the threads between the layers, the tab 32 will become substantially stiffer than the membrane as a whole. This can be used to advantage for securing the stent/membrane assembly in place in the blood vessel after anastomosis.

Namely, it may occur that the stent and the membrane are displaced relative to each other once they have been inserted in the blood vessel joint. One reason for this to happen is that the two blood vessels to be joined in the anastomosis procedure do not always exhibit exactly the same diameter. When this is the case, i.e. the diameters differ slightly the tension in the larger vessel will of course become less than in the smaller vessel which will be more tightly positioned on the stent. Such differences in tension may give rise to a force vector in the longitudinal direction of the stent, and can cause a dislocation or displacement of the stent or the membrane.

Manipulations during surgery will of course mechanically affect the stent/membrane assembly which can cause the mentioned dislocation to occur.

Thus, in operation of the assembly according to this embodiment, the stent will be placed such that the tab 32 is positioned in the actual joint, extending out therefrom. When the thread 20 subsequently is released, thereby causing the stent to expand, it will be an easy matter to pick up the thread at a point adjacent the attachment on the tab 32 by e.g. a pair of forceps and then pull the thread 20 such that the loose end 25 runs around the stent assembly. When the entire thread has been pulled out it can be secured in the tissue e.g. by suturing, and the excess length of thread is simply cut off. In this way the membrane is secured from being dislocated. In particular, if the inner end 8 of the membrane 6 is attached to the stent, the stent will also be secured by this operation and cannot move.

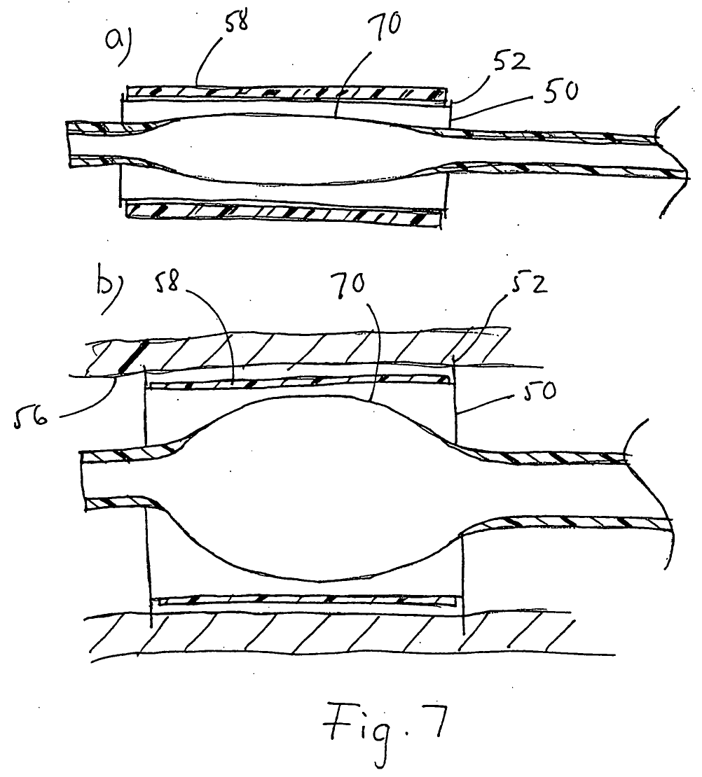

In another embodiment of the invention, the stent and membrane assembly is usable together with a balloon catheter for dilatation purposes. In this embodiment the stent need not be made of a shape memory material, i.e. it need not be self expandable. However, it must of course still exhibit the ability to assume both a contracted and an expanded state. For this application the stent carrying the membrane is positioned over the balloon on a catheter. The entire assembly is inserted into an artery to the location where a dilatation is desired. By inflating the balloon, the stent will expand and the membrane will follow the expansion and cover the stent by slightly more than one turn. FIG. 7a and 7b schematically illustrates use with a balloon catheter in a non-inflated state (FIG. 7a), and an inflated state (FIG. 7b). The balloon is designated 70.

In order to be usable on a catheter that is to be inserted into the blood vessel tree structure, as far as into the coronary vessels, it must be possible to keep the membrane in place on the stent during the insertion operation. It would not suffice to just wrap the membrane around the stent, since it would easily slide off from the correct position due to the friction encountered inside the blood vessels.

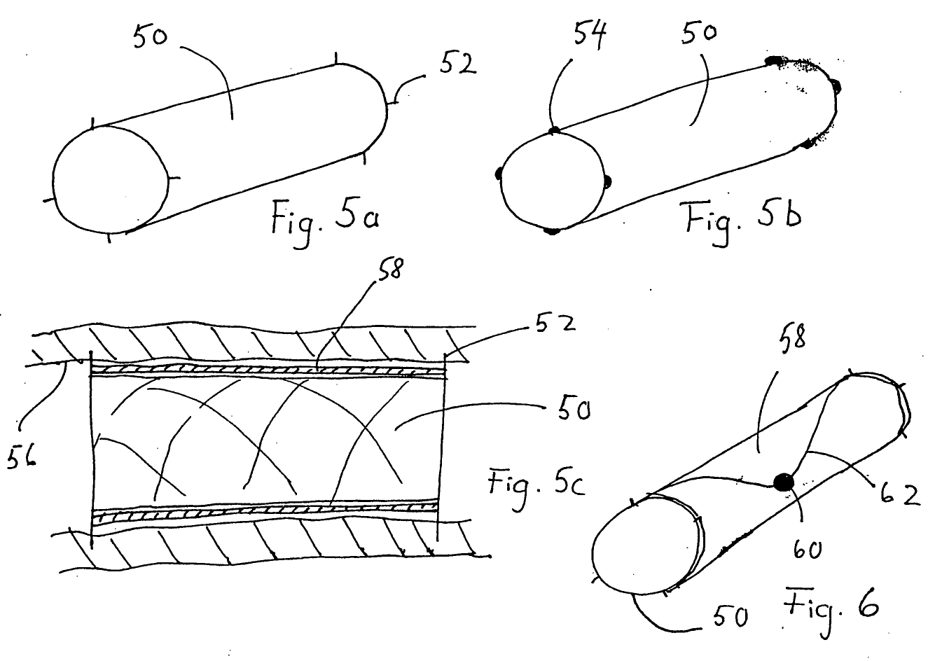

Therefore, as can be seen in FIG. 5a, which is a schematic perspective view of a generally cylindrical stent 50, there is provided a number of end stops 52, at least two, preferably three or more (four shown in FIG. 5a), distributed along the periphery of the stent at its ends. These end stops can be provided in several ways. In one version the thread material (suitably metal) from which the stent is made, can be used to provide very small upwardly directed, protruding pins or pegs. Alternatively, there could be provided pellet like structures 54, see FIG. 5b. If the stent is made from a moldable material such as a polymer, the pins can be provided in the molding of the stent itself.

The end stops would have an extension in the radial direction that does not exceed the thickness of the wrapped membrane, in order not to present any obstacles during the insertion operation. For example, a typical thickness of a membrane is about 50 μm, and in a contracted stat of the stent, the membrane typically is wrapped three turns around the stent. Thus, the total thickness of the membrane would be about 150 μm (In FIG. 7a, the several turns of the membrane 58 are shown as one layer for clarity). Typically, the end stops would protrude about 100 μm outwards, such that when the stent has expanded, and the membrane is wrapped only one turn (plus some overlap), the membrane would still be kept in place longitudinally by the provision of said end stops, as can be seen in FIG. 7b.

Furthermore, once the stent is expanded, the end stops could be made to extend above the surface of the membrane. Thereby, the pins or pegs can act as hooks, actually gripping the inner wall 56 of the blood vessel, thereby effectively keeping the stent 50 in place, preventing it from being displaced in the longitudinal direction of the vessel, see FIG. 5c and FIG. 7b. At the same time the end stops continue to act as such, i.e. they will prevent the membrane 58 from being slid off the surface of the stent.

In the case of the embodiment described earlier, wherein the stent is made of a shape memory material (for use as a connector), the stent can be given a memory of the expanded state in which the pins or pegs have a radial extension, thereby acting as hooks in the same way as just described, but in the contracted state, i.e. when cooled down, the pins will assume a longitudinally extended position, or even slightly bent inwards. In this way, the stent can be easily inserted into the vessel ends without the pins obstructing the passage. When put in place in the vessel the stent will expand. When the vessel ends are brought together for suturing, the pins will have changed their direction (i.e. regained the “remembered” configuration) and will grip the inner wall of the vessel, thereby securing the stent in correct position.

For the embodiment usable with balloon dilatation catheters, the membrane 58 must be secured in its wrapped condition on the stent 50. This can be ascertained by attaching a glue dot 60 on the outer end edge of the membrane, so as to fix the edge against the membrane surface. In the case of a triangularly shaped end edge 62, the apex of the triangle would suitably constitute the point of attachment, see FIG. 6.

The stent/membrane assembly can be made by the following procedure:

The surfaces of the stent and the membrane respectively are provided with surface bound heparin, by methods known per se by the skilled man. One example is disclosed in our own Swedish patent 9102798-7 (corresponding to U.S. Pat. No. 5,529,986).

Other surface treatments are also possible and within the inventive concept. It may for example be desirable to provide various kinds of medicaments on the surfaces of the membrane and of the stent, respectively, for selective local administration at the outside surface of the membrane facing vascular tissue. Examples of such medicaments are immunosuppressive agents and anti-proliferative agents.

The stent is cooled e.g. by cold spray to a small diameter, say 2 mm. Then, a rectangular piece of the membrane material, preferably having a triangularly shaped outer end is wound so as to form a cylinder with an inner diameter sized so as to accommodate the contracted stent. The triangular end must be located on the surface of the membrane cylinder. If desired the cylinder shape can be fixated by a heat treatment of the membrane cylinder, before placing the crimped stent therein.

Then the handle 14 or manipulator piece is set up to receive the cylindrical assembly 2. Preferably the handle has the locking thread 20 provided as a loop of a size larger than the diameter of the stent/membrane assembly, extending out from the aperture 22 located in the support surface 18 for the stent/membrane assembly 2. The cylindrical assembly is then placed on the support surface 18 within the loop 20.

The cold and contracted stent is positioned inside the lumen of the membrane cylinder, and the locking thread is pulled tight and the ends are secured. In this way the assembly will be maintained in a contracted state, and can be subjected to packaging and sterilization procedures to render it ready to use.

In the case where the membrane is attached to the stent, the procedure for assembling the device will differ slightly, but still comprises cooling of the stent. However, of course the membrane will be wound around the stent, and the thread is tightened to keep the crimped state.

A vessel anastomosis may be performed as follows:

The two vessels to be joined are clamped by vascular clamps. Two lateral and one posterior stay sutures are applied, for the purpose of pulling the ends together once the stent has been positioned appropriately.

The stent device is directed by means of the handle into one of the free vessel ends to half its length as set by the position of the handle. Then the other vessel end is passed over the opposite end of the stent assembly and the stay sutures are tied. Alternatively, the three stay sutures may be tied to join the free ends of the two vessels before implanting the stent device. When the stent is in place, an anterior suture is added. Now, the locking thread keeping the stent in a crimped or contracted state is released, and the stent and the membrane will expand simultaneously to the size of the vessel. The membrane provides immediate and complete sealing, and also prevents a traumatising contact between the stent and the tissue. The thread is then pulled away and the handle is easily removed from the anastomosis site. The vessel clamps are released and removed to re-establish blood circulation.

Thus, by means of the new device a tight and non-leaking connection of two blood vessels can be achieved by a very simple and quick procedure, which also ascertains an excellent sealing, which is leak-proof immediately upon completion of the connection. A further advantageous effect implied by rapid closure and restoration of blood flow, is that the ischemic time is substantially reduced, which in turn reduces the hazards involved when performing e.g. transplantation surgery.

In fact, the time for performing an anastomosis procedure on a laboratory animal (pig) was reduced from about 20-30 minutes using a traditional method down to 8-10 minutes employing the device and method according to the invention.

It will be obvious that the invention as described may be varied in many ways. Such variations are not to be regarded as a deviation from the spirit and scope of the invention, and all such modifications as would be obvious to one skilled in the art are intended to be included within the scope of the following claims.

1. A stent assembly (2), comprising
a stent (4; 50) capable of assuming a contracted and an expanded configuration;
a membrane (6; 58) of a biocompatible material impermeable to molecular transport across the membrane wrapped around said stent (4; 50) more than one full turn;
characterized in that
the membrane (6; 58) is made of a laminated material, comprising at least two layers with different thermal expansion coefficients.

2. The stent assembly as claimed in claim 1, wherein the end portions (8, 10) of the membrane are free relative to each other and to the stent.

3. The stent assembly as claimed in claim 1, wherein the inner end portion (8) of the membrane is attached (9) to the surface of the stent (4).

4. The stent assembly as claimed in claim 1, wherein the length of said membrane is such that the free ends thereof overlap also when said stent has been allowed to expand.

5. The stent as claimed in claim 1, wherein the surfaces of the stent and of the membrane are chemically or biologically modified.

6. The stent assembly as claimed in claim 5, wherein the surface modification comprises immobilisation of heparin on the surface.

7. The stent assembly as claimed in claim 5, wherein one or more of the surfaces on the stent/membrane assembly is provided with a medicament for selective local administration.

8. The stent assembly as claimed in claim 7, wherein the medicaments are selected from immunosuppressive agents and anti-proliferative agents.

9. The stent assembly as claimed in claim 1, wherein the outer end (10) of the membrane (6; 58) has a triangular shape (21; 62).

10. The stent assembly as claimed in claim 10, wherein the apex of the triangular end (21) is provided with a tab (32) onto which the thread (20) is attached such that a loop (34) is formed, said loop extending out from the tab (32).

11. The stent assembly as claimed in claim 1, wherein the stent is made of a shape memory material.

12. The stent assembly as claimed in claim 1, wherein the stent is expandable from a contracted state by the force from a balloon provided on a catheter, whereby the stent is provided over said balloon.

13. The stent assembly as claimed in claim 1, wherein the end edges of the stent (50) are provided with a plurality of end stop means (52; 54) distributed around the periphery of the edges, for keeping the membrane (58) in position on the stent (50).

14. The stent assembly as claimed in claim 13, wherein the end stop means are formed from the same thread material as the stent is made of, and constitute pins (52) pointing outwardly essentially in the radial direction.

15. The stent assembly as claimed in claim 13, wherein the end stop means are formed as pellet (54) like structures.

16. The stent assembly as claimed in claim 13, wherein the stent is made of a moldable material, and the end stop means are formed during molding of the stent.

17. A device for applying a stent assembly, comprising
a stent assembly (2) as claimed in claim 1 in a contracted state;
a holding and gripping device (12) comprising
a handle portion (14) and
a support structure (16, 18) for accommodating said stent assembly
a locking loop (20) that can be tightened around the stent assembly when it is positioned on said support structure, and released to allow said stent assembly to expand to a nominal size.

18. The device as claimed in claim 12, wherein said loop is formed by a thread (20) extending through said handle, and protrudes out from said support structure (16, 18) through an aperture (22) in the surface (18) of said support structure.

19. The stent assembly as claimed in claim 2, wherein the length of said membrane is such that the free ends thereof overlap also when said stent has been allowed to expand.

20. The stent assembly as claimed in claim 3, wherein the length of said membrane is such that the free ends thereof overlap also when said stent has been allowed to expand.
